(19) United States
(12) Patent Application Publication
(10) Pub. No.: US 2017/0079979 A1
(43) Pub. Date: Mar. 23, 2017

(54) THERAPY FOR SOLID TUMORS

(71) Applicant: Children's Hospital Medical Center, Cincinnati, OH (US)

(72) Inventors: Mohammad Azam, Mason, OH (US); Meenu Kesarwani, Mason, OH (US)

(21) Appl. No.: 15/312,329

(22) PCT Filed: May 29, 2015
(86) PCT No.: PCT/US15/33269

§ 371 Date: Nov. 23, 2016

Related U.S. Application Data
(60) Provisional application No. 62/006,808, filed on Jun. 2, 2014.

(51) Int. Cl. A61K 31/517; A61K 31/437; A61K 31/404; A61K 31/12; A61K 31/135

(52) U.S. Cl. CPC A61K 31/517; A61K 31/12; A61K 31/135; A61K 31/404; A61K 31/437

(57) ABSTRACT

A pharmaceutically acceptable composition and method for solid tumor therapy in a patient in need of such therapy. The composition contains, as the only active agents, the combination of (a) an inhibitor of c-Fos, and (b) an inhibitor of Dusp-1, and optionally (c) an inhibitor of a tyrosine kinase. The composition is administered to the patient in a dosing regimen for a period sufficient to provide therapy for solid tumors.

This application claims priority to U.S. Provisional Application Ser. No. 61/006,808 filed Jun. 2, 2014.

The invention was made with government support under CA155091 awarded by the National Institutes of Health. The government has certain rights in the invention.

A composition and method of using the composition to effect therapy for a solid tumor is provided. Therapy for targeting cancer stem cells are included. As used herein, therapy and treatment broadly encompass disease cure, or any lessening of disease presence, prevalence, severity, symptoms, etc.

In one embodiment, the composition contains at least one biocompatible excipient and, as its only active agents, the combination of at least one inhibitor of c-Fos, and at least one inhibitor of Dusp-1. In one embodiment, the composition further comprises at least one inhibitor of a tyrosine kinase. In various embodiments, the tyrosine kinase inhibitor targets epidermal growth factor receptor (EGFR), HER2/neu (human EGFR type 2), B-Raf, and/or targets multiple receptor tyrosine kinases (RTKs) such as platelet-derived growth factor receptor (PDGF-R), vascular endothelial growth factor receptor (VEGFR), KIT (CD117), RET, colony stimulating factor receptor (CSF-1R), and FLT3 cytokine receptor. In one embodiment, the composition contains at least one biocompatible excipient and, as its only active agents, the combination of one inhibitor of c-Fos, one inhibitor of Dusp-1, and one inhibitor of a tyrosine kinase. In any of the aforementioned embodiments, the inhibitor may inhibit the gene and/or the protein, i.e., the c-Fos inhibitor may inhibit the c-Fos gene and/or protein, the Dusp-1 inhibitor may inhibit the Dusp-1 gene and/or protein, and the tyrosine kinase inhibitor may inhibit the tyrosine kinase gene and/or protein. Such inhibitors include commercially available inhibitors and inhibitors under development. Small molecule inhibitors, such as curcumin, difluorinated curcumin (DFC), [3-{5-[4-(cyclopentyloxy)-2-hydroxybenzoyl]-2-[(3-hydroxy-1,2-benzisoxazol-6-yl) methoxy]phenyl}propionic acid] (T5224, Roche), nordihydroguaiaretic acid (NDGA), dihydroguaiaretic acid (DHGA), [(E,E,Z,E)-3-methyl-7-(4-methylphenyl)-9-(2,6,6-trimethyl-1-cyclohexen-1-yl)-2,4,6,8-nonatetraenoic acid (SR11302, Tocris Biosciences), (E)-2-benzylidene-3-(cyclohexylamino)-2,3-dihydro-1H-inden-1-one (BCI), TPI-2, TPI-3, triptolide, lapatinib, erlotinib, sunitinib, and vemurafenib (PLX4032) are encompassed. In one embodiment, inhibitors of c-Fos used in the composition are curcumin, difluorinated curcumin (DFC), [3-{5-[4-(cyclopentyloxy)-2-hydroxybenzoyl]-2-[(3-hydroxy-1,2-benzisoxazol-6-yl) methoxy]phenyl}propionic acid] (T5224, Roche), nordihydroguaiaretic acid (NDGA), dihydroguaiaretic acid (DHGA), and [(E,E,Z,E)-3-methyl-7-(4-methylphenyl)-9-(2,6,6-trimethyl-1-cyclohexen-1-yl)-2,4,6,8-nonatetraenoic acid (SR11302, Tocris Biosciences). In one embodiment, inhibitors of Dusp-1 are (E)-2-benzylidene-3-(cyclohexylamino)-2,3-dihydro-1H-inden-1-one (BCI), also known as NSC 150117, TPI-2, TPI-3, and triptolide. In one embodiment, inhibitors of tyrosine kinase are lapatinib, erlotinib, sunitinib, and vemurafenib (PLX4032).

In one embodiment, a composition and method is provided for treatment of a solid tumor that is dependent on HER2/neu and/or EGFR pathways, such as breast cancer. Lapatinib is a dual tyrosine kinase inhibitor that interrupts the HER2/neu and EGFR pathways. In one embodiment, the composition administered is curcumin and BCI. In one embodiment, the composition administered is curcumin, BCI, and lapatinib. In one embodiment, the composition administered is difluorinated curcumin (DFC) and BCI. In one embodiment, the composition administered is difluorinated curcumin (DFC), BCI, and lapatinib. In one embodiment, the composition administered is NDGA and BCI. In one embodiment, the composition administered is NDGA, BCI, and lapatinib. In one embodiment, the composition administered is T5224 and BCI. In one embodiment, the composition administered is T5224, BCI, and lapatinib. In one embodiment, the composition is administered to the patient at a concentration of 2 grams per day to 8 grams per day inclusive of the c-Fos inhibitor, 100 mg per day to 600 mg per day, inclusive of BCI, and when present, 400 mg to 800 mg per day inclusive of the tyrosine kinase inhibitor lapatinib 1500 mg daily. The composition is alkaline, about pH 8.5.

In one embodiment, a composition and method is provided for treatment of a solid tumor that is dependent on EGFR pathways, such as lung cancer. Erlotinib is a tyrosine kinase inhibitor that acts on EGFR. In one embodiment, the composition administered is curcumin and BCI. In one embodiment, the composition administered is curcumin, BCI, and erlotinib. In one embodiment, the composition administered is difluorinated curcumin (DFC) and BCI. In one embodiment, the composition administered is difluorinated curcumin (DFC), BCI, and erlotinib. In one embodiment, the composition administered is NDGA and BCI. In one embodiment, the composition administered is NDGA, BCI, and erlotinib. In one embodiment, the composition administered is T5224 and BCI. In one embodiment, the composition administered is T5224, BCI, and erlotinib. In one embodiment, the composition is administered to the patient at a concentration of 2 grams per day to 8 grams per day inclusive of the c-Fos inhibitor, 100 mg per day to 600 mg per day inclusive of BCI, and when present, 400 mg to 800 mg per day inclusive of the tyrosine kinase inhibitor erlotinib 150 mg once daily. The composition is alkaline, about pH 8.5.

In one embodiment, a composition and method is provided for the treatment of a solid tumor that is dependent on platelet-derived growth factor receptor (PDGF-R) pathways, such as lung sarcoma. Sunitinib is a tyrosine kinase inhibitor that targets multiple receptor tyrosine kinases (RTKs), such as PDGF-R, vascular endothelial growth factor receptor (VEGFR), KIT (CD117), RET, colony stimulating factor receptor (CSF-1R), and FLT3 cytokine receptor. In one embodiment, the composition administered is curcumin and BCI. In one embodiment, the composition administered is curcumin, BCI, and sunitinib. In one embodiment, the composition administered is difluorinated curcumin (DFC) and BCI. In one embodiment, the composition administered is difluorinated curcumin (DFC), BCI, and sunitinib. In one embodiment, the composition administered is NDGA and BCI. In one embodiment, the composition administered is NDGA, BCI, and sunitinib. In one embodiment, the composition administered is T5224 and BCI. In one embodiment, the composition administered is T5224, BCI, and sunitinib. In one embodiment, the composition is administered to the patient at a concentration of 2 grams per day to 8 grams per day inclusive of the c-Fos inhibitor, 100 mg per day to 600 mg per day inclusive of BCI, and when present, 400 mg to 800 mg per day inclusive of the tyrosine kinase inhibitor sunitinib 50 mg once daily. The composition is alkaline, about pH 8.5.

In one embodiment, a composition and method is provided for treatment of a solid tumor that is dependent on a mutation of B-Raf signaling pathways, such as bladder cancer. Vemurafenib (PLX4032) is a tyrosine kinase inhibitor that interrupts the B-Raf/MEK step on the B-Raf/MEK/ERK pathway, where B-Raf has the V600E mutation. In one embodiment, the composition administered is curcumin and BCI. In one embodiment, the composition administered is curcumin, BCI, and vemurafenib (PLX4032). In one embodiment, the composition administered is difluorinated curcumin (DFC) and BCI. In one embodiment, the composition administered is difluorinated curcumin (DFC), BCI, and vemurafenib (PLX4032). In one embodiment, the composition administered is NDGA and BCI. In one embodiment, the composition administered is NDGA, BCI, and vemurafenib (PLX4032). In one embodiment, the composition administered is T5224 and BCI. In one embodiment, the composition administered is T5224, BCI, and vemurafenib (PLX4032). In one embodiment, the composition is administered to the patient at a concentration of 2 grams per day to 8 grams per day inclusive of the c-Fos inhibitor, 100 mg per day to 600 mg per day inclusive of BCI, and when present, 400 mg to 800 mg per day inclusive of the tyrosine kinase inhibitor vemurafenib (PLX4032) 960 mg twice daily. The composition is alkaline, about pH 8.5.

In one embodiment, the composition is administered to the patient for 30 days. The composition may be administered by any route including but not limited to intravenous administration. The composition is preferably administered intravenously, orally, intramuscularly, transdermally, and/or intraperitoneally. Any biocompatible excipient may be used in the inventive composition, as known to one skilled the art. Biocompatible excipients include, but are not limited to, buffers, tonicity agents, pH modifying agents, preservatives, stabilizers, penetrant enhances, osmolality adjusting agents, etc. In one embodiment, the composition components are administered as individual components by the same route of administration or by different routes of administration, with administration of each component or components at substantially the same time. In one embodiment, the composition components are formulated into a cocktail, using methods known by one skilled in the art.

Cancer can be treated by identifying a molecular defect. This was demonstrated with chronic myelogenous leukemia (CML), a slow-growing bone marrow cancer resulting in overproduction of white blood cells. CML was the first cancer with a demonstrable association with a defined genetic abnormality: abnormal phosphorylation of cellular proteins by a deregulated enzyme, specifically, BCR-ABL tyrosine kinase.

A small molecule inhibitor, Imatinib mesylate (Gleevec™), was developed to block aberrant BCR-ABL tyrosine kinase activity. Gleevec™ was a major breakthrough in cancer therapy; Imatinib treatment revolutionized CML management and paved the way for development of tyrosine kinase inhibitor therapy for other diseases.

Despite Imatinib's efficacy in treating CML patients, it failed to provide a curative response because it preferentially targets the differentiated and dividing cells, therefore causing relapse upon Imatinib withdrawal. A limitation to develop curative therapy is lack of understanding of the molecular and patho-physiological mechanisms driving cancer maintenance, progression, mechanisms of therapeutic response and relapse. As with CML, differentiated and dividing cells undergo apoptosis following acute inhibition of BCR-ABL, termed “oncogene addiction”. In contrast, leukemic stem cells (LSCs) do not show a similar response. Given the intrinsic resistance of LSCs to TKI therapy in CML, the molecular mechanisms of oncogene addiction in therapeutically responsive cells allow targeting of the LSCs.

More specifically, the BCR-ABL tyrosine kinase inhibitor Imatinib improved the survival of patients with leukemia, but did not eliminate leukemia initiating cells (LIC). This suggested that LICs were not addicted to BCR-ABL.

As previously stated, Imatinib treatment is not curative. Many patients develop resistance despite continued treatment and some patients simply do not respond to treatment. Evidence suggests that a subset of cancer cells, termed “cancer stem cells”, drive tumor development and are refractory to most treatments. Cancer cells that do respond to drug treatment are critically dependent upon uninterrupted oncogene function, are “addicted to oncogene”; in contrast, cancer stem cells that are refractory to most treatments are not dependent on or “addicted to oncogene”. Eradicating cancer stem cells that are not “addicted to oncogene” is a critical part of successful anti-cancer therapy, and forms the basis of the invention.

A limitation to develop curative cancer therapy has been lack of understanding of the molecular and patho-physiological mechanisms driving cancer maintenance, progression, and mechanisms of therapeutic response and relapse. Oncogene addiction is the “Achilles' heel” of many cancers. In 2002, Weinstein proposed the concept that cancer cells acquire abnormalities in multiple oncogenes and tumor suppressor genes. Inactivation of a single critical gene can induce cancer cells to differentiate into cells with normal phenotype, or to undergo apoptosis, termed “oncogene addiction”. This dependence or addiction for maintaining the cancer phenotype can be exploited in cancer therapy.

In CML, differentiated and dividing cells undergo apoptosis following acute inhibition of BCR-ABL, and are thus “BCR-ABL addicted”. However, CML LICs do not show a similar response and are thus not “addicted” to BCR-ABL function. Solid tumor cancer stem cells are also believed to be refractory to treatment because they too are not “addicted” to the predominant tyrosine kinase function.

EGFR inhibitors in treatment of lung cancer represents another example of oncogene addiction that has yielded clinical success in a subset of patients with advanced disease that are otherwise refractory to conventional chemotherapy treatment. Mutations in the kinase domain of EGFR are found in a small subset of non-small cell lung cancers (NSCLC), and clinical responses to EGFR inhibitors, Gefitinib and Erlotinib have been well correlated with such mutations. Cancer genome sequencing data have also highlighted the likely role of “kinase addiction” in a variety of human cancers, e.g., activation of MET, BRAF, FGFR2, FGFR3, ALK, AURK and RET kinase in various different malignancies. Underscoring the importance of oncogene addiction is the fact that in all of these kinase-mediated malignancies, acute inactivation of the mutated kinase by either genetic or pharmacological means results in growth inhibition or tumor cell death. However, single TKI treatment is not curative. Therefore, simultaneously targeting the c-Fos and Dusp-1 that mediate addiction will eradicate the cancerous cells. In sum, the potential and importance of oncogene addiction in molecularly targeted cancer therapy highlights the fact that activated oncogenes, especially kinases, represent cancer culprits that frequently contribute to a state of oncogene dependency.

Cell culture models, genetically engineered mice, and clinical testing of targeted drugs support a widespread role for oncogene addiction in tumor cell maintenance and response to acute oncoprotein inactivation. The mechanism by which cells acquire dependency on a single pathway or activated protein is not clear in most cases, but multiple theories such as signaling network dysregulation, synthetic lethality genetic streamlining, and oncogenic shock have been postulated. Experimental evidence to prove these models is generally lacking, and it is unlikely that a single mechanism accounts for the numerous experimental findings that appear to represent examples of oncogene dependency. Mechanisms governing oncogene addiction may also vary according to the cellular and extracellular context.

Given the intrinsic resistance of CSCs to TKI therapy in solid tumors, a detailed understanding of oncogene dependency in therapeutically responsive cells permits engineering the therapeutically resistant cells CSCs to achieve drug sensitivity.

The inventors previously discovered that an inhibitor of c-Fos and an inhibitor of Dusp-1, in conjunction with an inhibitor of BCR-ABL tyrosine kinase, provided therapy for leukemia, which is a blood-born cancer usually comprising white blood cells and originating in the bone marrow. So far targeted therapy geared towards inhibiting the driver oncogene. As tumor types have different driver kinase therefore treatments were targeted to block oncogenic kinase signaling. This study for the first time revealed that signaling from the oncogenic kinases converge at c-Fos and Dusp1 and they are essential to establish the oncogenesis. Thus making these two targets more attractive as it seems targeting them along with TKI will allow us to eradicate almost all kinase driven cancers.

The present inventive method targets solid tumor cancer stem cells to produce curative therapies that do not require lifelong treatments.

In solid tumors, it had previously been shown that tyrosine kinase inhibitors did not eliminate cancer stem cells (CSCs) because these cells are not addicted to oncogene, even though tyrosine kinase inhibitors improved patient survival. The inventive method demonstrates that the down-regulation of c-Fos and Dusp-1 mediate tyrosine kinase addiction in TKI responsive tumor cells, and that inhibition of c-Fos and Dusp-1 together induces addiction in cancer stem cells, thus a combination of Dusp1 and c-Fos inhibitors will eliminate all cancerous cells. Given that the Dusp-1 and c-Fos knockout mice are viable and survive without any serious phenotype, suggesting these targets are suitable for therapeutic development. The inventive method assessed effectiveness of targeted c-Fos and Dusp-1 inhibition in CSCs for tyrosine kinase inhibitor response. This provided a basis for clinical application of a composition containing a c-Fos inhibitor, and a Dusp-1 inhibitor, and optionally a tyrosine kinase inhibitor, to target cancer cells, such as cancer stem cells (CSCs) of solid tumors.

### BRIEF DESCRIPTION OF THE DRAWINGS

FIG. 1 shows pharmacological inhibition of Dusp1 and c-Fos inhibits the growth factor mediated TKI resistance in AU656 cells (Breast Cancer-Her2 Amplified).

FIG. 2 shows pharmacological inhibition of Dusp1 and c-Fos inhibits the growth factor mediated TKI resistance in HCC827 cells (Lung Cancer-EGFR-M/Amplified).

FIG. 3 shows pharmacological inhibition of Dusp1 and c-Fos inhibits the growth factor mediated TKI resistance in H1703 cells (Lung Sarcoma-PDGFR Amplified).

FIG. 4 shows pharmacological inhibition of Dusp1 and c-Fos inhibits the growth factor mediated TKI resistance in RT4 cells (urinary bladder cancer has amplification of EGFR).

Cancer stem cells are intrinsically resistant to small-molecule kinase inhibitors. This discovery has prompted interest in developing strategies to more effectively target cancer initiating cells. One line of activity involves global gene expression analyses. Another line of activity involves identification of downstream partners essential for maximum tyrosine kinase oncoprotein activity. These have reinforced early evidence of activation of the JAK/STAT, PI3K/AKT, RAS/MAPK and NFKB pathways in cancer cells. These studies have also identified differentially expressed genes involved in regulation of DNA repair, cell cycle control, cell adhesion, homing, transcription factors, and drug metabolism. None of these studies identified potential therapeutic targets useful to eradicate the cancer stem cells. Based on these observations, knowing the mechanisms of oncogene addiction in TKI sensitive cells will permit determination of targets and co-therapeutic agents to achieve sensitivity for kinase inhibitors.

In one embodiment, inhibitors of tyrosine kinase are lapatinib, erlotinib, sunitinib, and vemurafenib (PLX4032).

In one embodiment, the Dusp-1 inhibitor is at least one of BCI, TPI-2, TPI-3, and triptolide. In one embodiment, the Dusp-1 inhibitor is BCI.

In one embodiment, the c-Fos inhibitor is at least one of curcumin, difluorinated curcumin (DFC), T5224, nordihydroguaiaretic acid (NDGA), dihydroguaiaretic acid (DHGA), and SR11302. In one embodiment, the c-Fos inhibitor is curcumin. In one embodiment, the c-Fos inhibitor is difluorinated curcumin (DFC). In one embodiment, the c-Fos inhibitor is NDGA. In one embodiment, the c-Fos inhibitor is T5224.

It has previously been shown that c-Fos and Dusp-1 play a role in BCR-ABL addiction in CML, as described in U.S. Patent Published Application No. 20140031356, which is incorporated by reference in its entirety, and summarized below.

An unbiased mRNA expression profiling was performed using BAF3 cells, which requires IL-3 for survival, expressing the BCR-ABL tyrosine kinase under a Tet-R responsive promoter that renders them IL-3-independent. BaF3 cells were used because it is homogeneous in terms of gene expression, and because BCR-ABL dependence is reversible. Specifically, in the presence of exogenous IL-3, BAF3 cells no longer depend on BCR-ABL for survival.

To define the differential expression of gene(s) in BCR-ABL addicted and non-addicted conditions, expression analysis was performed using total RNA from BaF3 cells, BaF3 cells expressing BCR-ABL conditionally in the presence and absence of exogenously added IL-3 and BaF3-BCR-ABL cells treated with Imatinib in the presence and absence of IL-3.

These data showed that AP-1 transcription factor c-Fos and dual specificity phosphatase-1 mediated the BCR-ABL addiction. Only three genes, Dusp-1, Dusp-10, and c-Fos, were down regulated in BCR-ABL addicted cells, while they were upregulated to 3-5 fold in non-addicted cells. This suggested their role in BCR-ABL dependence. The role of these three genes in mediating BCR-ABL addiction were evaluated; specifically, whether their down-regulation in non-addicted cells would sensitize them to Imatinib induced apoptosis.

c-Fos, Dusp-1 and Dusp-10 were knocked down using shRNA hairpin, and cell survival analysis was performed in the presence of 5 μM Imatinib, which typically kills addicted cells in 24 hrs at this concentration, and IL-3. Dusp-1 and c-Fos knockdown alone induced 30% and 40% sensitivity to Imatinib, respectively. Dusp-10 knock down did not show any significant sensitivity to Imatinib. This suggested that double knock down of c-Fos and Dusp-1 may sensitize the BCR-ABL cells fully. To test this, instead of using shRNA mediated gene knock down of Dusp-1, a small molecule inhibitor that targets Dusp-1, BCI, was used. In cell proliferation assays, BaF3-BCR-ABL cells with c-Fos knockdown were fully sensitive to Imatinib when combined with BCI. The same combinations of drugs had no effect on BCR-ABL positive and parental BaF3 cells, highlighting the response specificity.

Efficacy of Dusp-1 and c-Fos inhibition in mouse model of CML and CD34⁺ cells from CML patients was shown. The BCR-ABL tyrosine kinase inhibitor Imatinib improves the survival of patients but does not eliminate LICs. This suggested that these cells are not addicted to BCR-ABL. The data demonstrated that downregulation of c-Fos and Dusp-1 mediated BCR-ABL addiction. Inhibition of c-Fos and Dusp-1 together induced apoptosis in BCR-ABL positive cells following Imatinib treatment. The same combination has no effect on survival and apoptosis of parental BaF3 cells. Dusp-1 and c-Fos knockout mice were viable and survived without any serious phenotype, suggesting that these targets were suitable for therapeutic development. The effectiveness of c-Fos and Dusp-1 inhibition in LICs for Imatinib response was determined.

Although leukemia and solid tumors differ in many functional and characteristic ways, such as their dependence on angiogenesis, the present inventors examined the effect of c-Fos and Dusp-1 inhibitors on solid tumor cells, which are tyrosine kinase dependent. Inhibition of c-Fos and Dusp-1 in various cellular models of solid tumors, such as breast, lung, bladder, and melanoma, was shown to evaluate the inventive composition as a therapeutic agent.

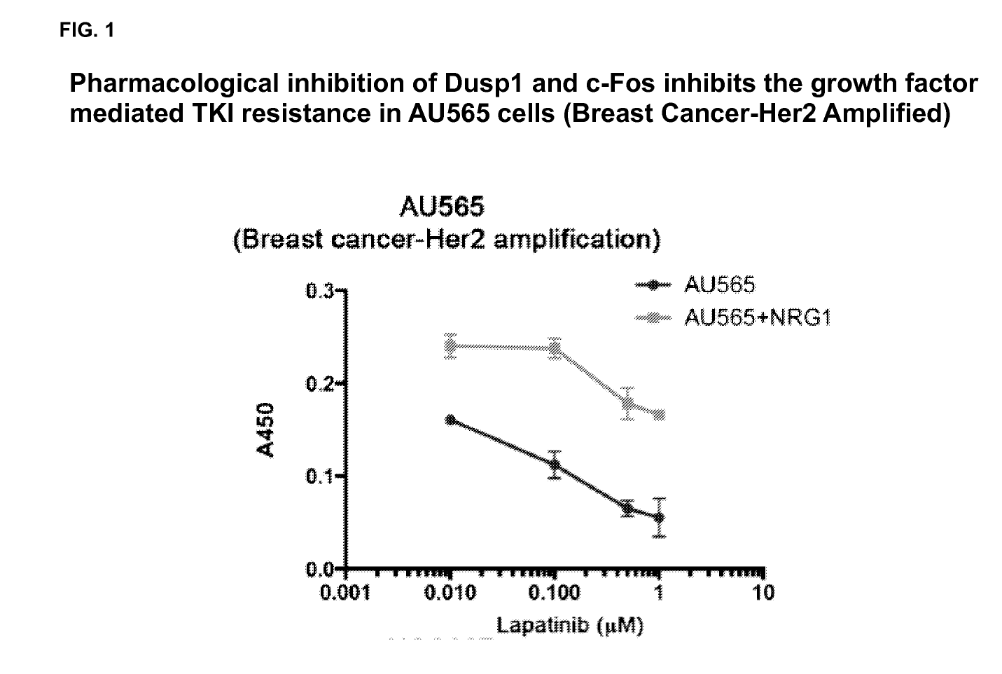

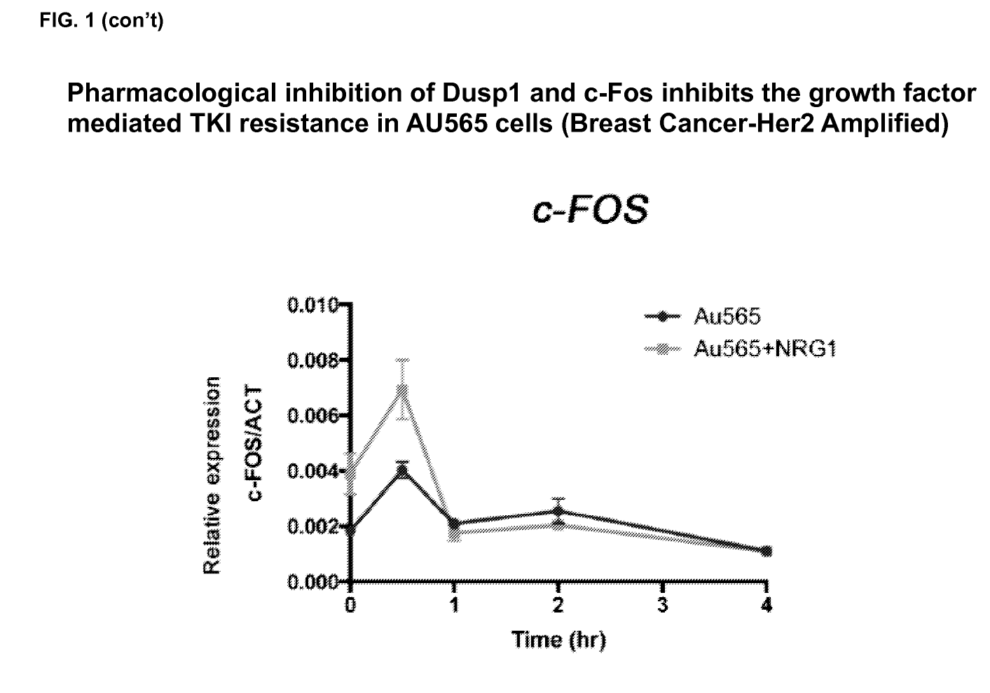

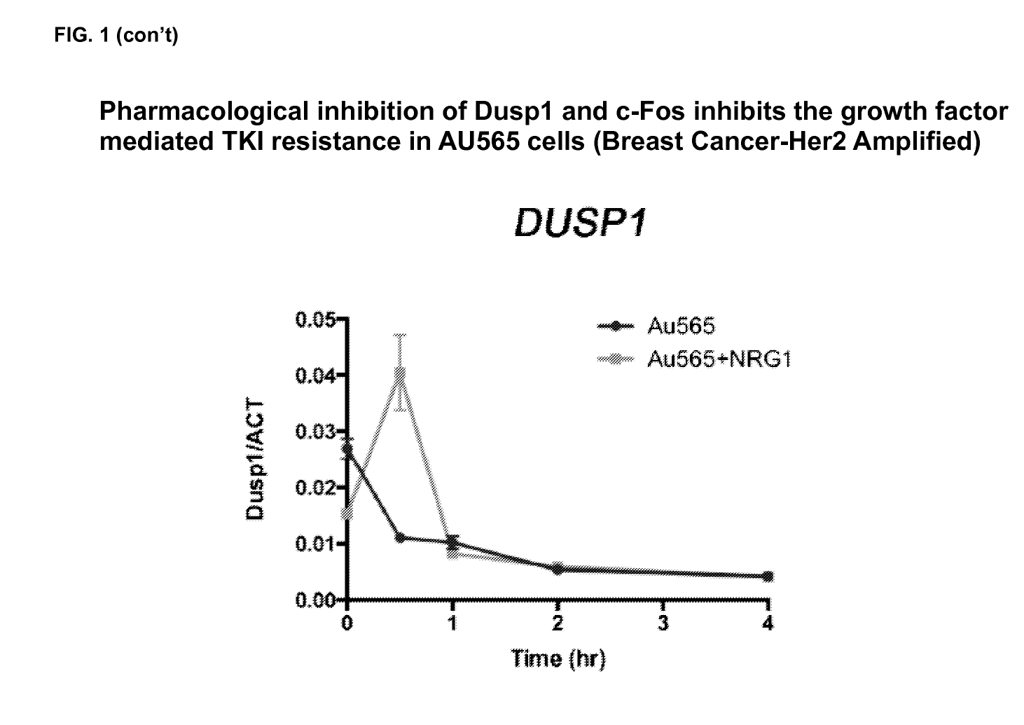

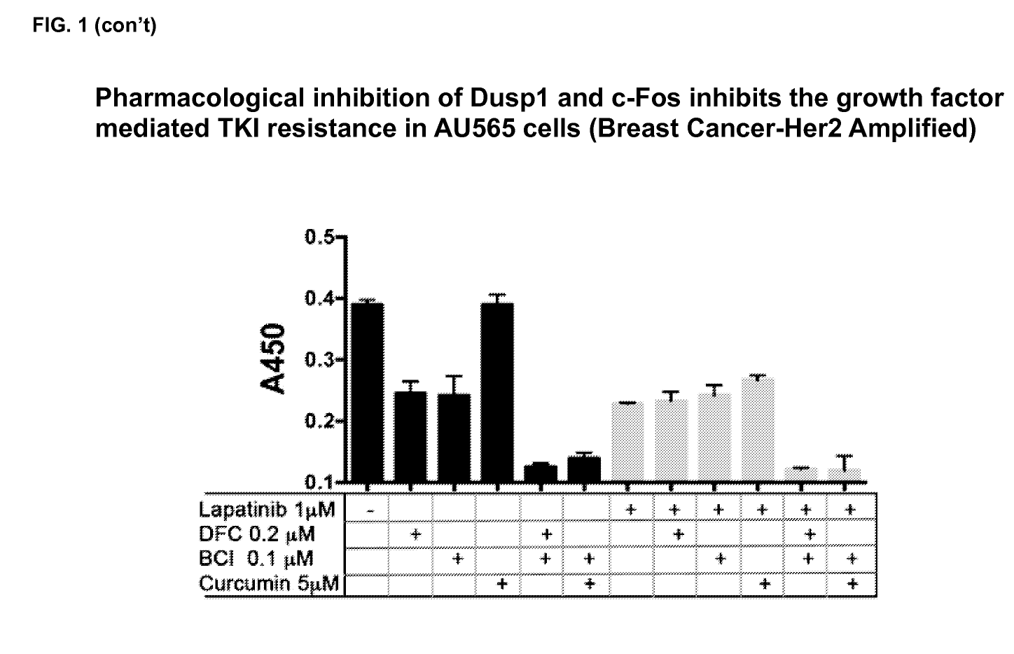

To determine the effect of c-Fos and Dusp-1 inhibition with the tyrosine kinase inhibitor lapatinib, AU565 cells were assayed, as a model of breast cancer, shown in FIG. 1. AU565 cell line was established from the malignant pleural effusion of the mammary gland, which overexpresses the HER2. This class of tumors is treated by lapatinib. This treatment is not curative and relapse is common because cancer stem cells are not responsive to the treatment. Recent studies demonstrate that growth factor NRG1 abrogates the TKI response in cancer stem cells. We show that the addition of NRG1 confer resistance to lapatinib, which seemingly mediated by the overexpression of DUSP-1 and c-FOS (FIG. 6). In support, we show that concomitant inhibition of Dusp-1 by BCI and c-Fos (either curcumin or DFC) completely suppresses the cell survival (FIG. 1, right panel), suggesting that the oncogenic signaling by HER2 is dependent on these two targets.

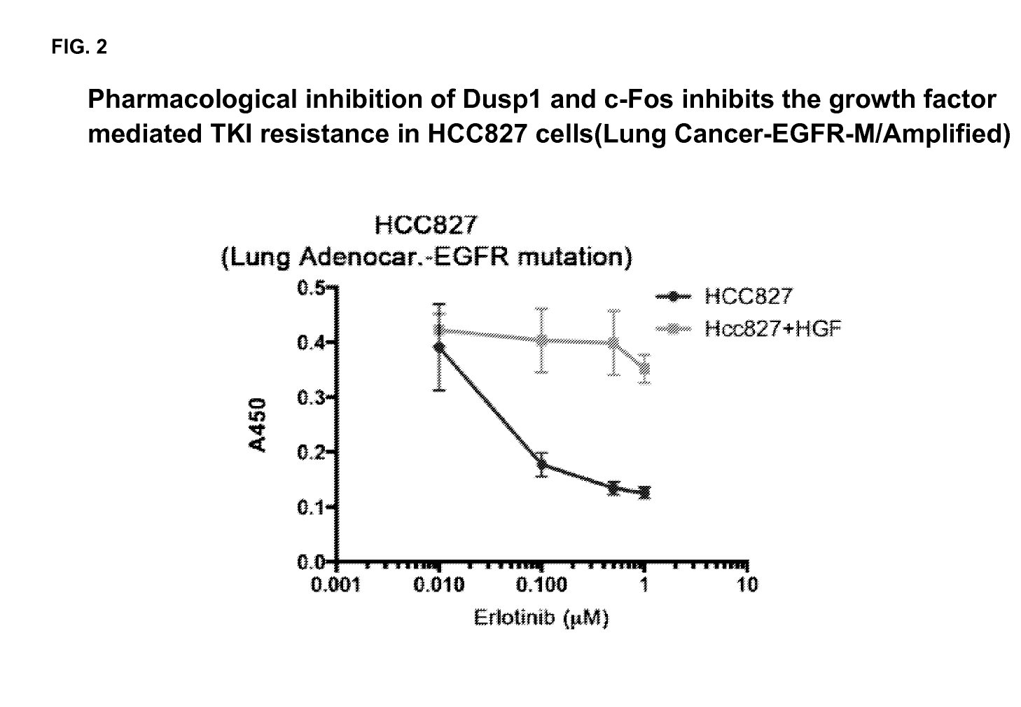

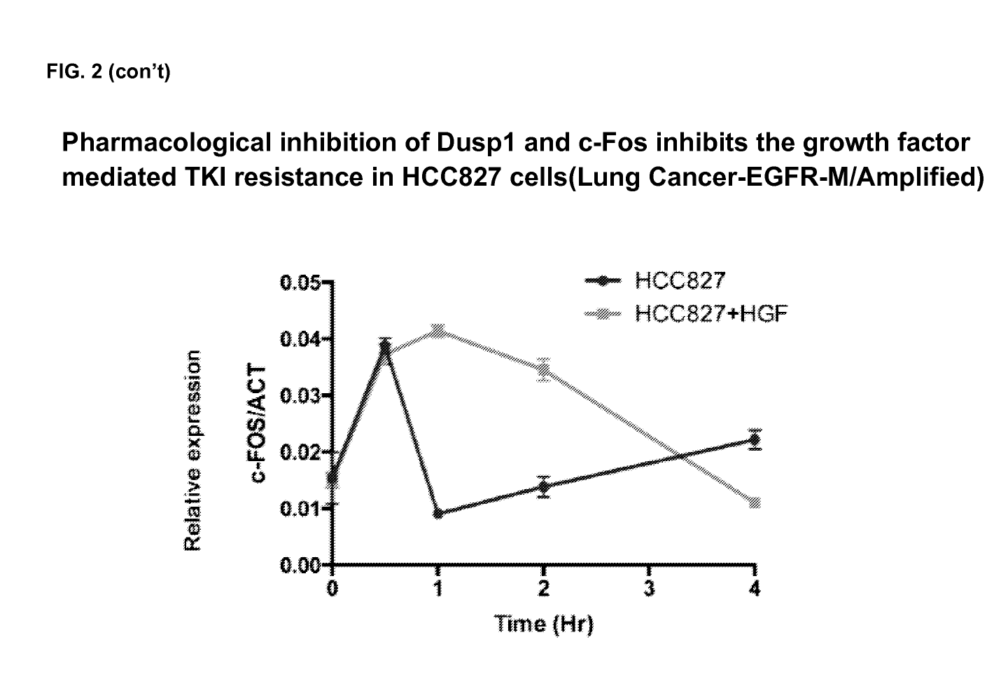

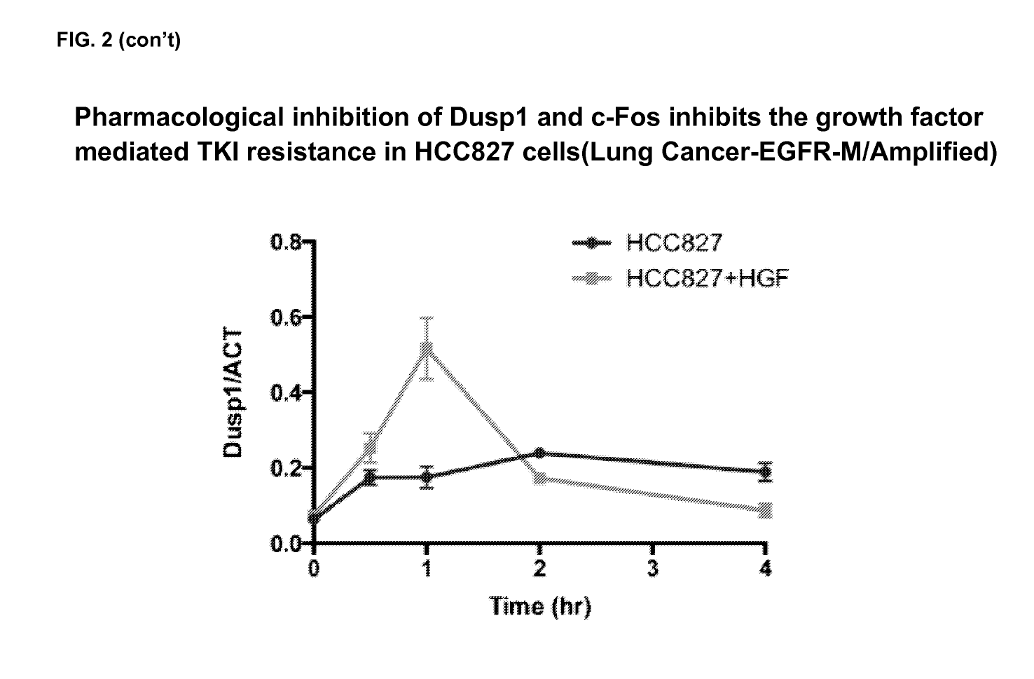

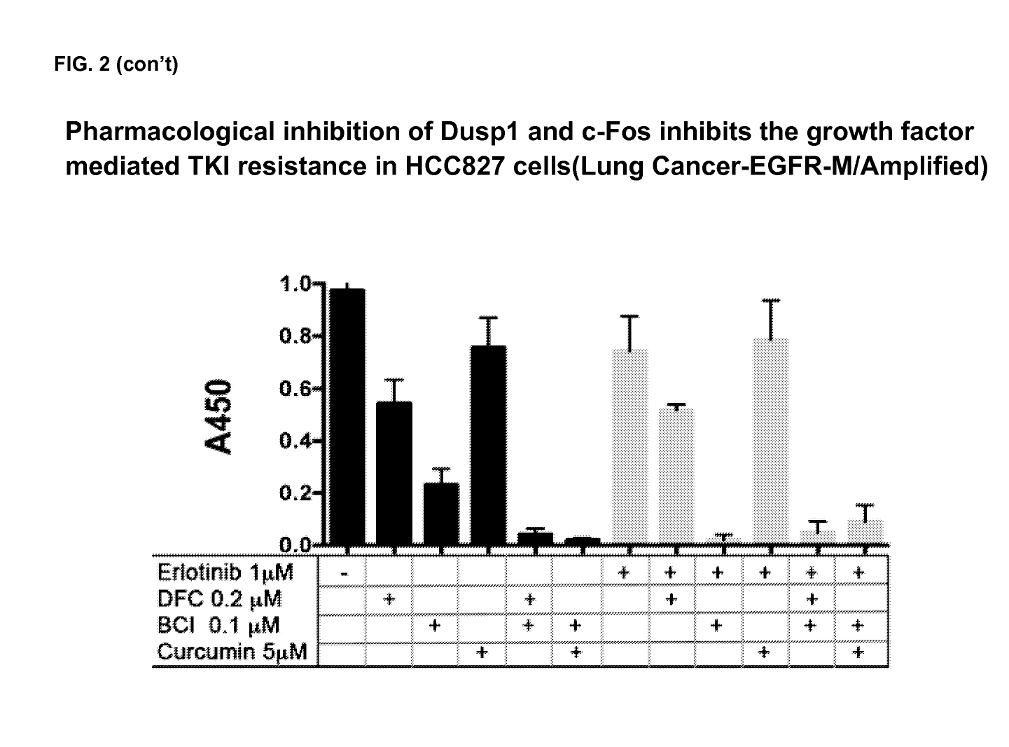

To determine the effect of c-Fos and Dusp-1 inhibition with the tyrosine kinase inhibitor erlotinib, a lung cancer HCC827 cells were assayed, as a model of lung cancer, shown in FIG. 2. HCC827 cell line was derived from the lung adenocarcinoma. This lung adenocarcinoma has an acquired mutation in the EGFR tyrosine kinase domain (E746-A750 deletion). Patients having EGFR kinase activating mutations are treated with either erlotinib or gefitinib. Most patients develop resistance by acquiring gatekeeper mutation the kinase domain and single TKI treatment is not curative because cancer stem cells are refractory to the treatment. In addition overexpression of growth factor HGF leading to activation of MET is a more frequent way of bypassing the EGFR mediated survival in this tumor model. Besides, cancer stem cells in the presence of growth factor are not addicted to the oncogene. We show that the addition of HGF confer resistance to erlotinib that also induces the the overexpression of DUSP-1 and c-FOS (FIG. 2). We also show that concomitant inhibition of Dusp-1 by BCI and c-Fos (either curcumin or DFC) completely suppresses the cell survival (FIG. 2, right panel), thus affirming the finding that the oncogenic signaling of EGFR is dependent on these two targets.

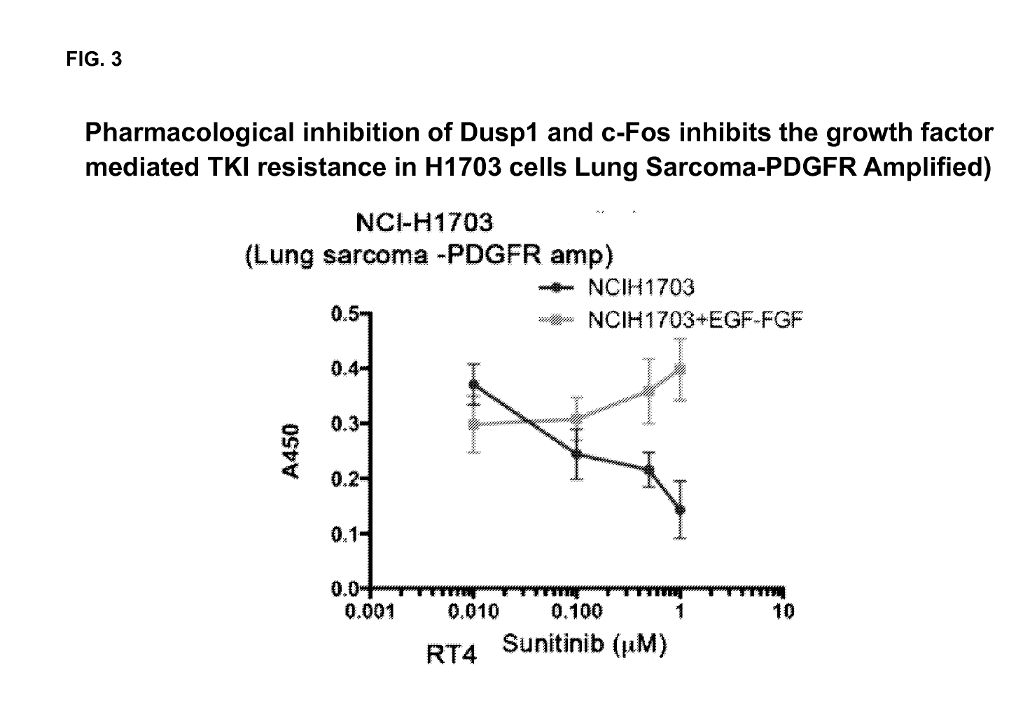

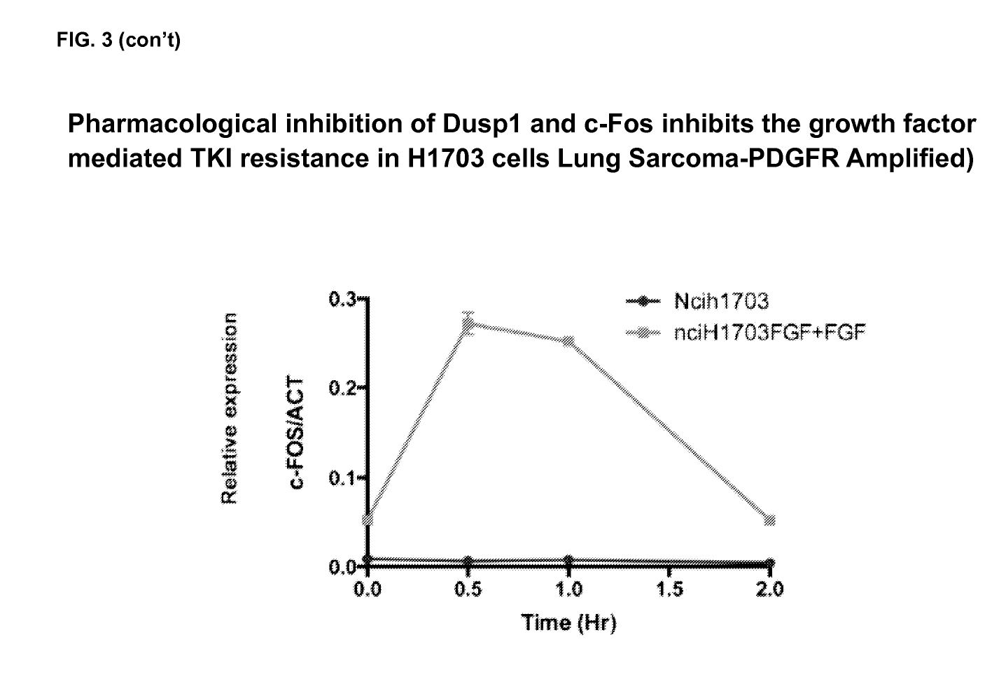

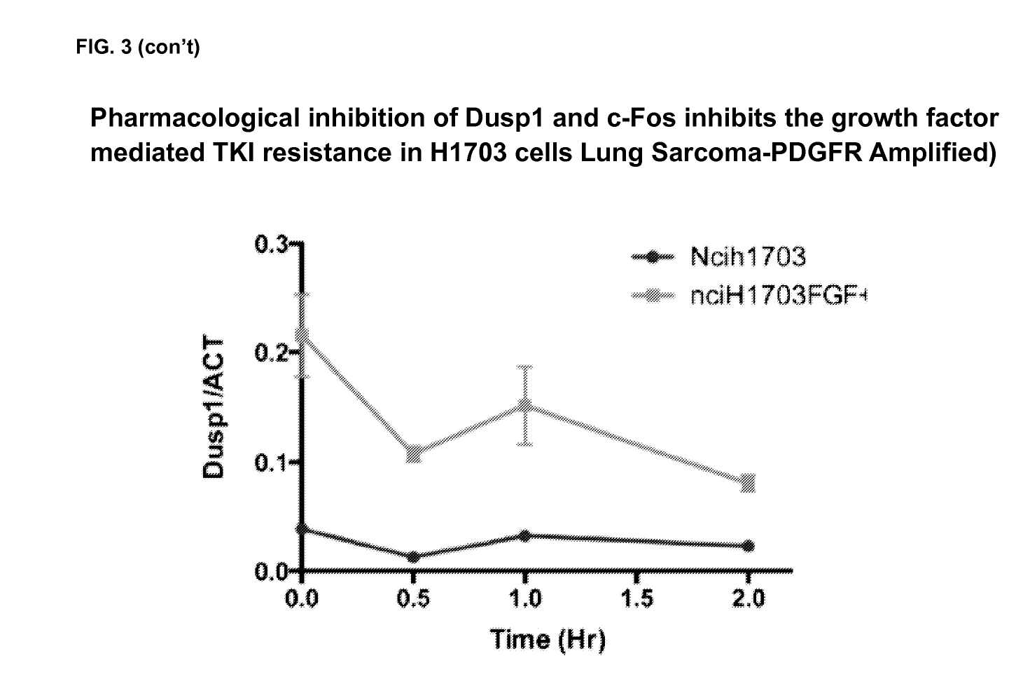

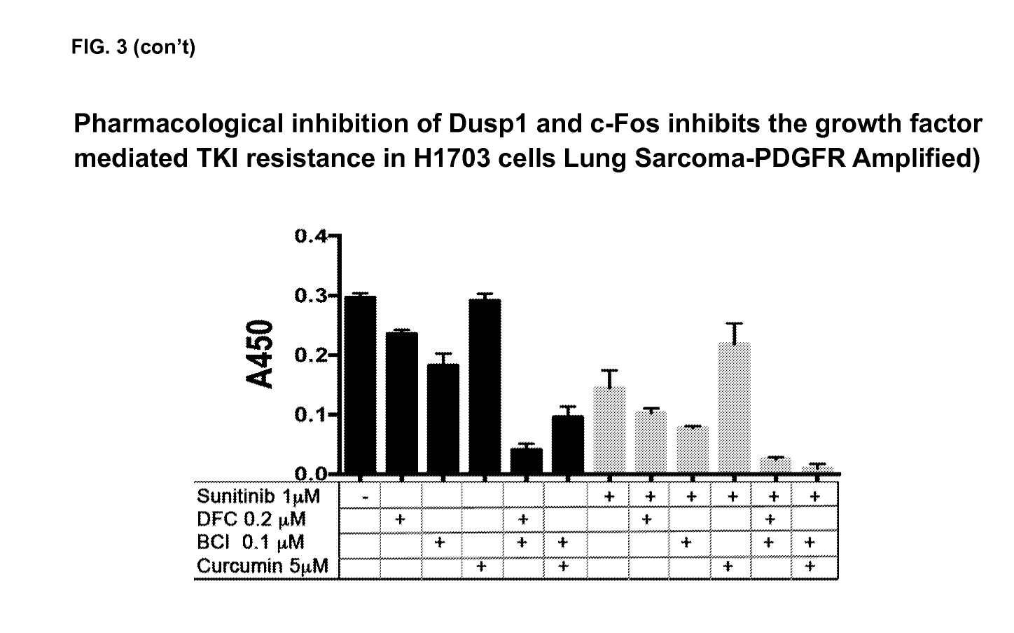

To determine the effect of c-Fos and Dusp-1 inhibition with the tyrosine kinase inhibitor sunitinib, H1703 cells were assayed, as a model of lung sarcoma, shown in FIG. 3. H1703 cell line was derived from the lung sarcoma, which is driven by PDGFRA as this cell line has amplified the PDGFRA. This group of patients is treated with sunitinib. Single TKI treatment is not curative because cancer stem cells are refractory to the treatment. In addition overexpression of growth factor EGF abrogates TKI response. We show that the addition of EGF confer resistance to sunitinib that also induces the overexpression of DUSP-1 and c-FOS (FIG. 3). We also show that the concomitant inhibition of Dusp-1 by BCI and c-Fos (either curcumin or DFC) completely suppresses the cell survival (FIG. 3, right panel), thus affirming the finding that the oncogenic signaling of EGFR is dependent on these two targets.

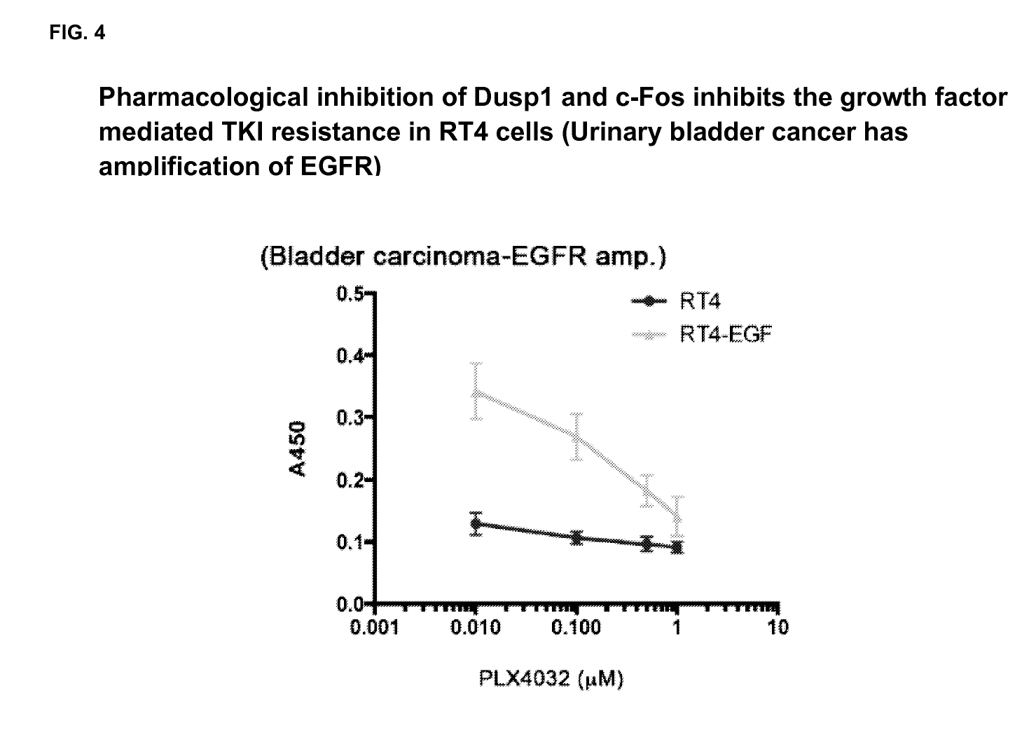

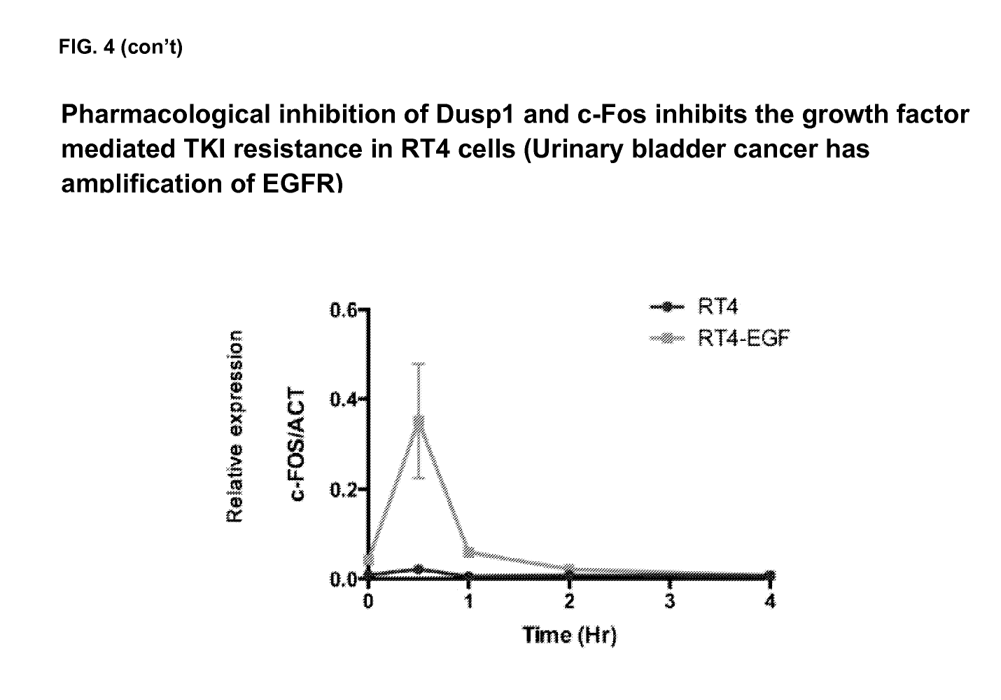

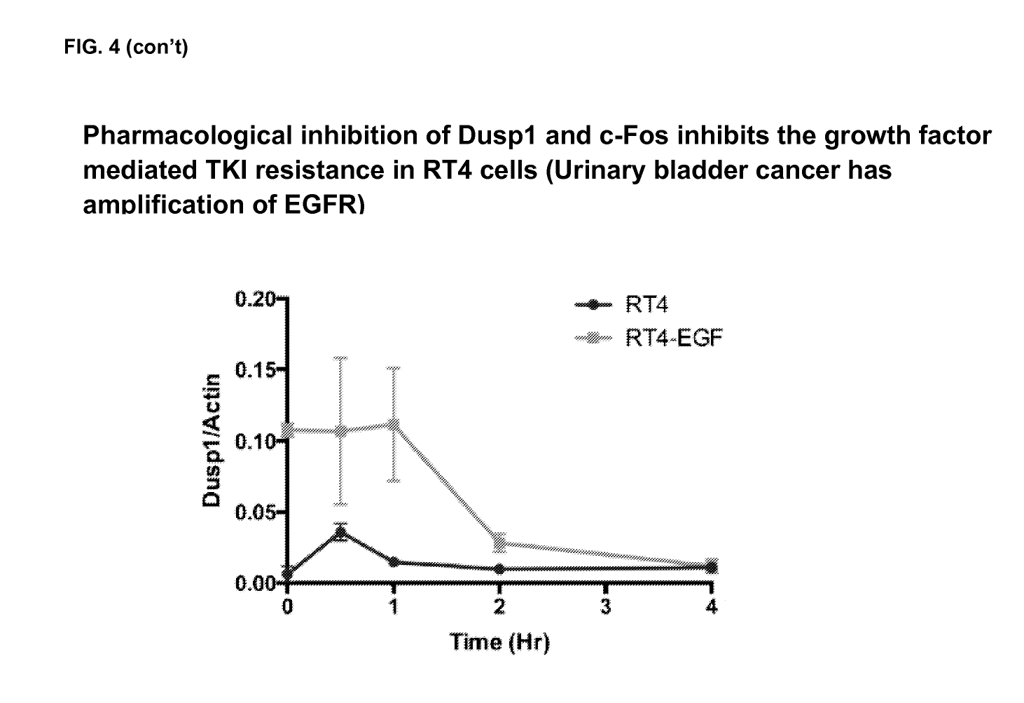

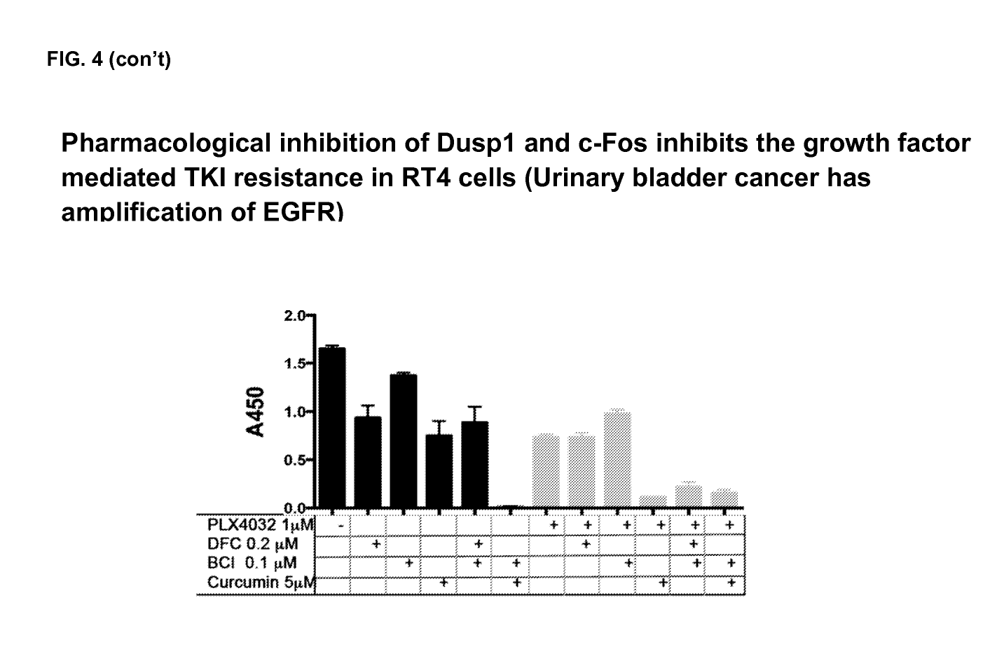

To determine the effect of c-Fos and Dusp-1 inhibition with the tyrosine kinase inhibitor PLX4032, RT4 cells were assayed, as a model bladder cancer, shown in FIG. 4. This cell line was derived from bladder cancer patients, which has amplification of EGFR. As reported, addition of EGF abrogates the TKI response. We show that the addition of EGF confers resistance to TKI that also induces the overexpression of DUSP-1 and c-FOS (FIG. 4). We also show that the concomitant inhibition of Dusp-1 by BCI and c-Fos (either curcumin or DFC) completely suppresses the cell survival (FIG. 4, right panel), where combination with curcumin is more potent. Thus suggesting that the oncogenic signaling of EGFR is dependent on these two targets in bladder cancer as well.

The above data demonstrated that a combination of c-Fos, Dusp-1, and tyrosine kinase inhibitors was more potent than the inhibitors alone or a combination of two inhibitors.

The in vitro data demonstrated that Dusp-1 and c-Fos mediated tyrosine kinase addiction in solid tumors, showing that Dusp-1 and c-Fos inhibitors are targets for curative therapy in solid tumors.

In one embodiment, a method is provided to treat solid tumor cancers. In various embodiments, the use of the described Dusp-1 and c-Fos inhibitors, either alone or in combination with a tyrosine kinase inhibitor (TKI), abrogated growth factor mediated resistance. In various embodiments, the solid tumors are kinase-driven solid tumors. Examples of such solid tumor cancers include breast cancer, lung cancer, and bladder. In one embodiment, a method comprising, or consisting essentially of, administering a described Dusp-1 and c-Fos inhibitors is provided, and is used in the treatment of breast cancer solid tumors. In one embodiment, a method comprising, or consisting essentially of, administering a described Dusp-1 and c-Fos inhibitors in combination with lapatinib is provided, and is used in the treatment of breast cancer solid tumors. In one embodiment, a method comprising, or consisting essentially of, administering a described Dusp-1 and c-Fos inhibitors is provided, and is used in the treatment of lung cancer solid tumors. In one embodiment, a method comprising, or consisting essentially of, administering a described Dusp-1 and c-Fos inhibitors in combination with erlotinib is provided, and is used in the treatment of lung cancer solid tumors. In one embodiment, a method comprising, or consisting essentially of, administering a described Dusp-1 and c-Fos inhibitors is provided, and is used in the treatment of lung sarcoma solid tumors. In one embodiment, a method comprising, or consisting essentially of, administering a described Dusp-1 and c-Fos inhibitors in combination with sunitinib is provided, and is used in the treatment of lung sarcoma solid tumors. In one embodiment, a method comprising, or consisting essentially of, administering a described Dusp-1 and c-Fos inhibitors is provided, and is used in the treatment of bladder solid tumors. In one embodiment, a method comprising, or consisting essentially of, administering a described Dusp-1 and c-Fos inhibitors in combination with PLX4032 is provided, and is used in the treatment of bladder solid tumors.

In one embodiment, the present composition is used to treat solid tumor cancers. In various embodiments, the use of the described Dusp-1 and c-Fos inhibitors, either alone or in combination with a tyrosine kinase inhibitor (TKI), abrogated growth factor mediated resistance. In various embodiments, the solid tumors are kinase-driven solid tumors. Examples of such solid tumor cancers include breast cancer, lung cancer, and bladder. In one embodiment, a composition comprising, or consisting essentially of, a described Dusp-1 and c-Fos inhibitors is provided, and is used in the treatment of breast cancer solid tumors. In one embodiment, a composition comprising, or consisting essentially of, a described Dusp-1 and c-Fos inhibitors in combination with lapatinib is provided, and is used in the treatment of breast cancer solid tumors. In one embodiment, a composition comprising, or consisting essentially of, a described Dusp-1 and c-Fos inhibitors is provided, and is used in the treatment of lung cancer solid tumors. In one embodiment, a composition comprising, or consisting essentially of, a described Dusp-1 and c-Fos inhibitors in combination with erlotinib is provided, and is used in the treatment of lung cancer solid tumors. In one embodiment, a composition comprising, or consisting essentially of, a described Dusp-1 and c-Fos inhibitors is provided, and is used in the treatment of lung sarcoma solid tumors. In one embodiment, a composition comprising, or consisting essentially of, a described Dusp-1 and c-Fos inhibitors in combination with sunitinib is provided, and is used in the treatment of lung sarcoma solid tumors. In one embodiment, a composition comprising, or consisting essentially of, a described Dusp-1 and c-Fos inhibitors is provided, and is used in the treatment of bladder solid tumors. In one embodiment, a composition comprising, or consisting essentially of, a described Dusp-1 and c-Fos inhibitors in combination with PLX4032 is provided, and is used in the treatment of bladder solid tumors.

Each of the following references is expressly incorporated by reference herein in its entirety:

Aikawa et al. “Treatment of arthritis with a selective inhibitor of c-Fos/activator protein-1,” Nature Biotechnology, vol. 26, no. 7 (2008), pp. 817-823.

Day et al., “Small Molecule Inhibitors of DUSP6 and Uses Therefor,” WO2010/108058, Sep. 23, 2010.

Park et al., “Inhibition of fos-jun-DNA complex formation by dihydroguaiaretic acid and in vitro cytotoxic effects on cancer cells,” Cancer Letters, vol. 127 (1998), pp. 23-28.

Padhye S, et al. New difluoro Knoevenagel condensates of curcumin, their Schiff bases and copper complexes as proteasome inhibitors and apoptosis inducers in cancer cells. Pharm Res 2009;26:1874-80.

Padhye S et al. Fluorocurcumins as cyclooxygenase-2 inhibitor: molecular docking, pharmacokinetics and tissue distribution in mice. Pharm Res. 2009 November;26(11):2438-45.

Other variations or embodiments will be apparent to a person of ordinary skill in the art from the above description. Thus, the foregoing embodiments are not to be construed as limiting the scope of the claimed invention.

What is claimed is:

1. A method of therapy for solid tumors in a patient, the method comprising administering to the patient in need thereof a composition containing at least one biocompatible excipient and, as the only active agents, a combination of
(a) an inhibitor of c-Fos, and
(b) an inhibitor of Dusp-1,
the composition administered to the patient in a dosing regimen for a period sufficient to provide therapy for solid tumors to the patient in need thereof.

2. The method of claim 1 further comprising administering to the patient in need thereof (c) an inhibitor of a tyrosine kinase.

3. The method of claim 1 where (a) is an inhibitor of a c-Fos gene, and (b) is an inhibitor of a Dusp-1 gene.

4. The method of claim 1 where (a) is an inhibitor of a c-Fos protein, and (b) is an inhibitor of a Dusp-1 protein.

5. The method of claim 2 wherein (c) is an inhibitor of a tyrosine kinase gene or an inhibitor of a tyrosine kinase protein.

6. The method of claim 1 where (a) is selected from the group consisting of curcumin, difluorinated curcumin (DFC), [3-{5-[4-(cyclopentyloxy)-2-hydroxpenzoyl]-2-[(3-hydroxy-1,2-benzisoxazol-6-yl) methoxy]phenyl}propionic acid] (T5224), nordihydroguaiaretic acid (NDGA), dihydroguaiaretic acid (DHGA), RE,E,Z,E)-3-methyl-7-(4-methylphenyl)-9-(2,6,6-trimethyl-1-cyclohexen-1-yl)-2,4,6,8-nonatetraenoic acid (SR11302), and combinations thereof; and (b) is selected from the group consisting of (E)-2-benzylidene-3-(cyclohexylamino)-2,3-dihydro-1H-inden-1-one (BCI), TPI-2, TPI-3, triptolide, and combinations thereof.

7. The method of claim 2 wherein (c) is selected from the group consisting of lapatinib, erlotinib, sunitinib, PLX4032, and combinations thereof.

8. The method of claim 2 where (a) is curcumin or DFC, (b) is BCI; and (c) is lapatinib.

9. The method of claim 2 where (a) is curcumin or DFC, (b) is BCI; and (c) is erlotinib.

10. The method of claim 2 where (a) is curcumin or DFC, (b) is BCI; and (c) is sunitinib.

11. The method of claim 2 where (a) is curcumin or difluorinated curcumin (DFC), (b) is BCI; and (c) is PLX4032.

12. The method of claim 2 where (a) is administered at a concentration of 2 grams per day to 8 grams per day, inclusive; (b) is administered at a concentration of 100 mg per day to 600 mg per day, inclusive; and (c) is administered at a concentration of 400 mg per day to 800 mg per day, inclusive.

13. The method of claim 1 where the composition is administered to the patient for 30 days.

14. The method of claim 1 where the composition is administered to the patient intravenously, orally, transdermally, intramuscularly, and/or intraperitoneally to result in an effective dosing regimen.

15. The method of claim 1 where the composition is administered as a cocktail.

16. The method of claim 1 where the patient has breast cancer, lung cancer, or bladder cancer.

17. The method of claim 1 wherein the patient has a kinase-driven solid tumor.

18-28. (canceled)

29. A pharmaceutically acceptable composition comprising at least one biocompatible excipient and, as the only active agents, (a) a c-Fos inhibitor selected from the group consisting of curcumin, diflourinated curcumin (DFC), [3-{5-[4-(cyclopentyloxy)-2-hydroxybenzoyl]-2-[(3-hydroxy-1,2-benzisoxazol-6-yl) methoxy]phenyl}propionic acid] (T5224), nordihydroguaiaretic acid (NDGA), dihydroguaiaretic acid (DHGA), and [(E,E,Z,E)-3-methyl-7-(4-methylphenyl)-9-(2,6,6-trimethyl-1-cyclohexen-1-yl)-2,4,6,8-nonatetraenoic acid (SR11302); and
(b) a Dusp-1 inhibitor selected from the group consisting of (E)-2-benzylidene-3-(cyclohexylamino)-2,3-dihydro-1H-inden-1-one (BCI—also known as NSC 150117), TPI-2, TPI-3, and triptolide.

30. The composition of claim 29 further comprising (c) a tyrosine kinase inhibitor selected from the group consisting of lapatinib, erlotinib, sunitinib, and PLX4032.

31. A pharmaceutically acceptable composition comprising at least one biocompatible excipient and, as the only active agents, (a) a c-Fos inhibitor, and (b) a Dusp-1.

32. The composition of claim 31 further comprising (c) a tyrosine kinase inhibitor.

33. A pharmaceutically acceptable composition comprising at least one biocompatible excipient and, as the only active agents, (E)-2-benzylidene-3-(cyclohexylamino)-2,3-dihydro-1H-inden-1-one (BCI), and difluorinated curcumin (DFC).

34. The composition of claim 33 further comprising lapatinib, erlotinib, sunitinib, or PLX4032.
